(19) United States
(12) Patent Application Publication
(10) Pub. No.: US 2014/0029808 A1
(43) Pub. Date: Jan. 30, 2014

(54) Body Condition Score Determination for an Animal

(71) Applicant: ClicRweight, LLC, Tampa, FL (US)

(72) Inventor: Ken Lee, Fairfax, VA (US)

(73) Assignee: ClicRweight, LLC, Tampa, FL (US)

(21) Appl. No.: 13/948,826

(22) Filed: Jul. 23, 2013

Related U.S. Application Data
(60) Provisional application No. 61/674,582, filed on Jul. 23, 2012.
(60) Provisional application No. 61/857,012, filed on Jul. 22, 2013.

(51) Int. Cl. G06K 9/00

(52) U.S. Cl. CPC G06K 9/00362
USPC 382/110

(57) ABSTRACT

Described are methods and systems for determining a body condition score (BCS) for an animal. An imaging device captures at least one image of an animal, where each image includes a body region of the animal, and transmits the image to a computing device. The computing device identifies the body region contained in the image and crops a portion of the image associated with the identified body region from the image. The computing device compares the cropped portion of the image with one or more fitting models corresponding to the body region and determines a body condition score for the body region based upon the comparing step.

### RELATED APPLICATIONS

This application claims priority to U.S. Provisional Patent Application No. 61/674,582, filed on Jul. 23, 2012, and to U.S. Provisional Patent Application No. 61/857,012, filed on Jul. 22, 2013, the entirety of which are incorporated herein by reference.

### TECHNICAL FIELD

The subject matter of the application relates generally to livestock management, and more particularly to processes and systems, including computer program products, for determining a body condition score for an animal based on analysis of at least one image of the animal.

### BACKGROUND

The body condition score (BCS) of an animal indicates the fatness or thinness of an animal according to a scale. This information is used to help determine the productivity, reproduction, health, and longevity of an animal. While there is no uniform scale that has been adopted world-wide, there are certain nationally accepted standards used by farmers in various regions. However, scoring for the different standards tends to be subjective and can vary between persons who are doing the scoring. In addition, it takes a significant amount of time and cost to implement a robust and efficient BCS tracking system.

### SUMMARY

Therefore, what is needed is a computer vision system and corresponding methods that can be used to automate the determination of a BCS to provide a lower cost, yet more reliable, measurement.

The invention, in one aspect, features a method for determining a body condition score (BCS) for an animal. An imaging device captures at least one image of an animal, where each image includes a body region of the animal, and transmits the image to a computing device. The computing device identifies the body region contained in the image, and crops a portion of the image associated with the identified body region from the image. The computing device compares the cropped portion of the image with one or more fitting models corresponding to the body region, and determines a body condition score for the body region based upon the comparing step.

The invention, in another aspect, features a system for determining a body condition score (BCS) for an animal. The system includes an imaging device configured to capture at least one image of an animal, where each image includes a body region of the animal, and transmit the image to a computing device coupled to the imaging device. The computing device is configured to identify the body region contained in the image, and crop a portion of the image associated with the identified body region from the image. The computing device is configured to compare the cropped portion of the image with one or more fitting models corresponding to the body region, and determine a body condition score for the body region based upon the comparing step.

The invention, in another aspect, features a computer program product, tangibly embodied in a non-transitory computer readable medium, for determining a body condition score (BCS) for an animal. The computer program product includes instructions operable to cause an imaging device to capture at least one image of an animal, where each image includes a body region of the animal, and transmit the image to a computing device coupled to the imaging device. The computer program product includes instructions operable to cause the computing device to identify the body region contained in the image, and crop a portion of the image associated with the identified body region from the image. The computer program product includes instructions operable to cause the computing device to compare the cropped portion of the image with one or more fitting models corresponding to the body region, and determine a body condition score for the body region based upon the comparing step.

Any of the above aspects can include one or more of the following features. In some embodiments, the computing device validates a posture of the animal in the image. The validating step includes generating a 3D point cloud based upon the image, performing one or more edge analysis tests on the 3D point cloud, and determining whether the posture is valid based upon the one or more edge analysis tests. In some embodiments, performing one or more edge analysis tests includes generating a cubic polynomial curve based upon the topmost points of the 3D point cloud, and analyzing the inflection point and concavity of the cubic polynomial curve. In some embodiments, the computing device analyzes the local minimum and maximum of the cubic polynomial curve.

In some embodiments, performing one or more edge analysis tests includes generating a linear model based upon the topmost points of the 3D point cloud, and analyzing the slope of the linear model. In some embodiments, the computing device determines that the posture is not valid if the slope of the linear model exceeds 0.12.

In some embodiments, each image includes a plurality of body regions. In some embodiments, identifying the body region contained in the image includes determining a type of the animal in the image, and selecting a portion of the image corresponding to the body region based upon the animal type. In some embodiments, the animal type includes a sex of the animal, a breed of the animal, and a species name of the animal.

In some embodiments, comparing the cropped portion of the image with one or more fitting models corresponding to the body region includes retrieving the one or more fitting models from a database based upon a type of the animal and the body region, identifying one or more anatomical features in the cropped portion of the image, identifying one or more anatomical features in the fitting models, and comparing cloud points in the cropped portion of the image that are associated with the one or more anatomical features with cloud points in the one or more fitting models that are associated with the one or more anatomical features. In some embodiments, each of the one or more fitting models is associated with a known BCS. In some embodiments, the comparing cloud points step includes determining an error value between the cloud points in the cropped portion of the image and the cloud points in the one or fitting models, and selecting the fitting model that has the minimum error value. In some embodiments, the determining an error value step is based upon an iterative closest point algorithm. In some embodiments, the computing device assigns the BCS associated with the fitting model that has the minimum error value to the cropped portion of the image.

In some embodiments, the computing device adjusts at least one of height, length or depth of at least one cloud point of (i) the image relative to one of the fitting models or (ii) one of the fitting models relative to the image. In some embodiments, the computing device determines whether the density of cloud points in the image meets a predefined threshold.

In some embodiments, the image is a 3D scan. In some embodiments, the body region is a rump, hip, backbone, thigh, short-rib, long-rib, tail-head, or pin bone of the animal. In some embodiments, the BCS is a score on a scale that indicates fatness or thinness of the animal. In some embodiments, where each image includes a plurality of body regions, the computing device determines overall body condition score for the animal based upon the body condition score for each of the plurality of body regions.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a block diagram of a system for determining a body condition score for an animal based on analysis of at least one image of the animal.

FIG. 2 is a flow diagram of a method for determining a body condition score for an animal based on analysis of at least one image of the animal.

FIG. 3 is a detailed flow diagram of a method for determining a body condition score for an animal based on analysis of at least one image of the animal.

FIG. 4 is a flow diagram of a method for validating the posture of the animal appearing the scan.

FIG. 5 is a diagram of an exemplary cubic polynomial curve model.

FIG. 6 is a diagram of an exemplary linear model.

FIG. 7 is a diagram of two different results produced by the computing device after conducting the linear model matching.

FIG. 8A is a diagram of an exemplary inflection point and concavity test on a cubic polynomial curve where the computing device determined that the inflection point and concavity measurements of the curve pass the test.

FIG. 8B is a diagram of an exemplary inflection point and concavity test on a cubic polynomial curve where the computing device 108 determined that the concavity measurements of the curve fail the test.

FIG. 8C is a diagram of an exemplary inflection point and concavity test on a cubic polynomial curve where the computing device determined that the inflection point measurements of the curve fail the test.

FIG. 9A is a diagram of an exemplary critical point test on a cubic polynomial curve where the computing device determined that the curve has at least one critical point and that the local maximum and local minimum exist within the x range of the point cloud and the local maximum is higher than the local minimum, thereby passing the test.

FIG. 9B is a diagram of an exemplary critical point test on a cubic polynomial curve where the computing device determined that the curve does not have any critical points, thereby failing the test.

FIG. 9C is a diagram of an exemplary local minimum test on a cubic polynomial curve where the computing device determined that the local minimum of the curve falls outside the x range of the point cloud, thereby failing the test.

FIG. 10 is a diagram of exemplary body regions of an animal (e.g., a cow) to be identified by the computing device for determination of a BCS.

FIG. 11 is a flow diagram of a method for comparing the scan of a region of interest for an animal with a fitting model for that region of interest to determine a BCS.

FIG. 12A is a side view diagram of a comparison between a 3D scan and a fitting model using an iterative closest point algorithm.

FIG. 12B is a top-down view diagram of a comparison between a 3D scan and a fitting model using an iterative closest point algorithm.

FIG. 13 is a diagram of a comparison between the half-lines of a 3D scan of an animal and a fitting model.

### DETAILED DESCRIPTION

The technology described herein features systems and methods for determining a BCS of an animal based on analysis of a 3D image of the animal which can be used to measure not just height, width, and depth of the animal but also the size of different body regions such as the rump or ribs, for instance. A database of animal “fitting” models with known BCS that were previously captured can be used as references for comparison to the captured 3D image of the animal. To determine a BCS, a body region of the animal is adjusted and compared to the reference fitting models until it fits the model with the corresponding BCS.

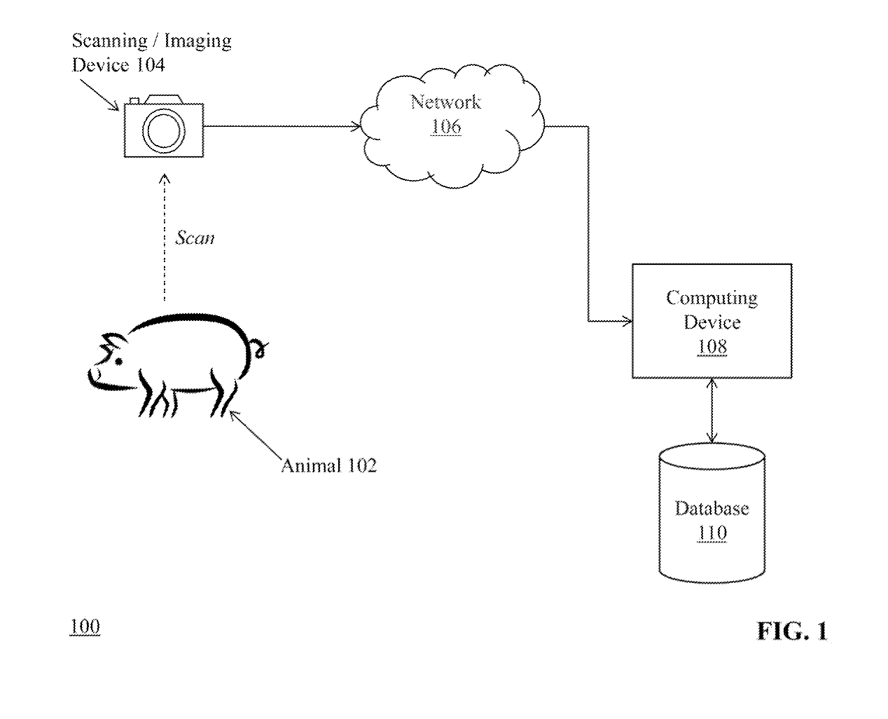

FIG. 1 is a block diagram of a system 100 for determining a body condition score for an animal based on analysis of at least one image of the animal. The system 100 includes an animal 102 (e.g., a pig), a scanning/imaging device 104, a communications network 106, a computing device 108, and a database 110. The methods described herein may be achieved by implementing program procedures, modules and/or software executed on, for example, a processor-based computing devices or network of computing devices.

The animal 102 is placed in proximity to the scanning/imaging device 104 so that the scanning/imaging device 104 captures an image or scan of the animal 102. While a pig is depicted in FIG. 1, it should be understood that other animals can be used within the scope of invention, including but not limited to cows, heifers, bulls, steers, pigs, hogs, sheep, goats, horses, other livestock, and/or dogs.

The imaging/scanning device 104 can be, e.g., a stereoscopic imaging device or an infrared camera. In some embodiments, multiple images of the animal are captured at one or more different angles. The imaging/scanning device 104 can include one or more filters, lenses or control mechanisms (e.g., auto-positioning, focusing or processing systems). The imaging/ scanning device 104 can be a stereoscopic video camera, a 3D scanner, a charged coupled device, a photodiode array, a CMOS optical sensor, a still photographic camera, a digital camera, and/or a conventional two-dimension camera. Multiple imaging/scanning devices can be used in some embodiments.

In some embodiments, the imaging/scanning device 104 includes a light source for illuminating a field-of-view of the device. The light source can be a coherent source, such as a laser, or an incoherent source, such as a light emitting diode. The light source can be configured to illuminate a broadside of the animal or to backlight the animal. The light source can be a linear array, such as an array of monochromatic light emitting diodes (LEDs) with diffusers. In some embodiments, the imaging/scanning device 104 includes a depth sensor such as an infrared laser projector combined with a monochrome CMOS sensor, which captures video data in 3D under ambient light conditions.

In some embodiments, the technology can be implemented in a closed-ended chute including a control wall having an animal feeder, an animal presence indicator and an imaging device having a field-of-view substantially unobstructed by walls of the chute. The implementation can include a control system communicatively connected to the animal presence indicator and the imaging device, and configured to control the imaging device based upon information communicated by the animal presence indicator.

The communications network 106 transmits captured images and scans to the computing device 108. The network 106 may be a local network, such as a LAN, or a wide area network, such as the Internet or the World Wide Web. The network 106 may utilize cellular, satellite or other wireless communications technology. For example, the scanning/imaging device 104 may send and receive information via a communications link to a satellite, which in turn communicates with the computing device 108.

The computing device 108 receives the captured images and scans from the scanning /imaging device 104 via the network 106. As will be described in greater detail below, the computing device 108 processes the images and scans to determine a BCS for the animal. The computing device 108 communicates with a database 110 for retrieval of fitting model data for comparison with the received scans and for storage of determined BCS data. In some embodiments, the computing device 108 is coupled to other computing devices (not shown). In some embodiments, the database 110 is internally integrated into the computing device 108. It should be appreciated that any number of computing devices, arranged in a variety of architectures, resources, and configurations (e.g., cluster computing, virtual computing, cloud computing) can be used without departing from the scope of the invention.

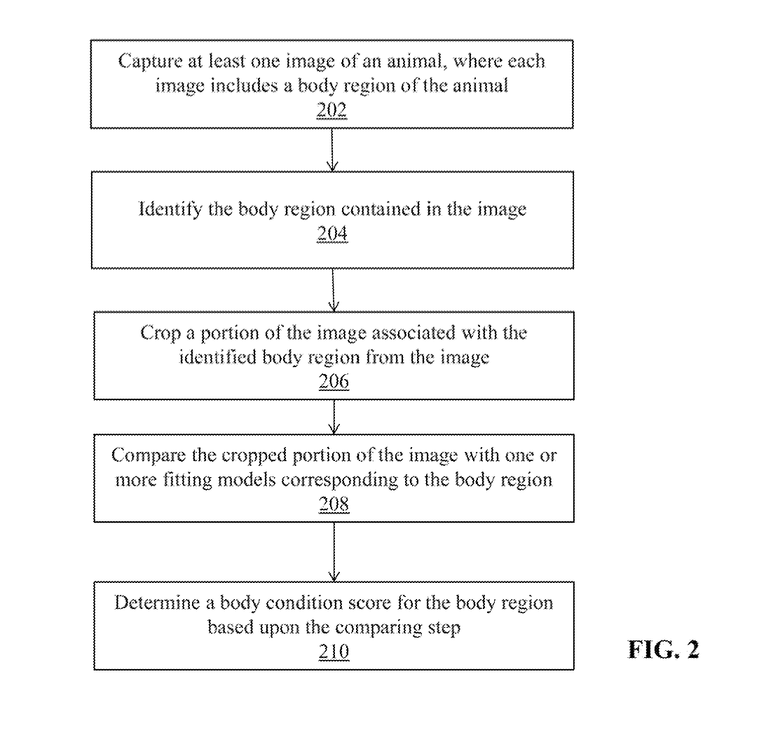

FIG. 2 is a flow diagram of a method for determining a body condition score for an animal based on analysis of at least one image or scan of the animal, using the system 100 of FIG. 1. Generally, the body condition score (BCS) is a score on a scale that indicates thinness or fatness of the animal. The fatness is directly related to the health of the animal and, in the case of a milk cow, its milk production. The score can range from 1 to 10, with 1 being the thinnest and 10 being the fattest.

The imaging/scanning device 104 captures (202) at least one image of an animal (e.g., animal 102), where the image includes a body region. In some embodiments, the imaging/scanning device 104 captures images of the animal from a plurality of different angles in order to accurately capture the shape of the animal—especially parts that are used to measure BCS.

The imaging/scanning device 104 transmits the captured image to the computing device 108 via the network 106. The computing device 108 identifies (204) each body region in the scan and crops (206) the body regions out of the scan as separate scans (called body region scans or regions of interest). For example, a rump area is identified and cropped out from the rest of the scan.

In some embodiments, the computing device 108 resizes each body region scan based upon a fitting model corresponding to the same body region. The fitting model is of a known size to make resizing more efficient.

The computing device 108 compares (208) each body region scan to the corresponding fitting models using an iterative closest point algorithm. The fitting model that returns a minimum error is considered to be a match for the body region scan. The computing device 108 determines (210) the BCS by assigning the BCS of the matching fitting model to the body region scan.

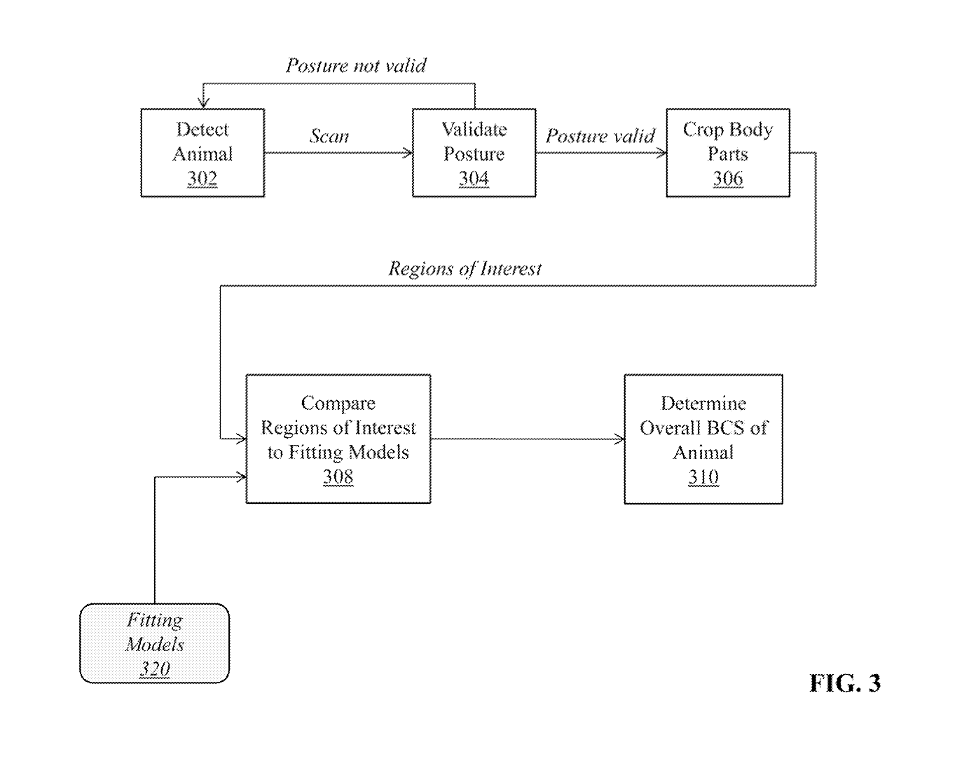

FIG. 3 is a detailed flow diagram of a method for determining a body condition score for an animal based on analysis of at least one image of the animal, using the system 100 of FIG. 1.

The imaging/scanning device 104 is always on and continuously captures images and/or scans of a specific area (e.g., a feeding pen). In some embodiments, the scans are captured at ten frames per second.

The imaging/scanning device 104 transmits each scan to the computing device 108, and the computing device 108 analyzes the scan to detect (302) whether an animal is present in the scan. For example, the computing device 108 compares the currently-received scan against the previous scan to determine whether any new objects appear within the current scan. The computing device can subtract the previous scan from the current scan, or determine whether the number of points within the current scan reflects a significant change (i.e., a greater number of points). If the computing device 108 does not detect the presence of an object, the device 108 does not conduct any further processing on the received scan.

If a new object is detected, the computing device 108 then extracts the newly-detected object from the background of the current scan. For example, the computing device 108 can subtract the region of the scan around the background points from the current scan, leaving the new object. In another example, the computing device 108 can grab the points around the region where the animal is expected to appear or stand.

The computing device 108 then compares the newly-detected object to a fitting model retrieved from the database 110 to determine whether the object does or does not contain any background points. To accomplish the comparison, the computing device 108 can analyze the scan using a curve-fitting algorithm such as Random Sample Consensus (RANSAC), or by using an iterative closest point (ICP) algorithm and determining a minimum ICP error. It should be understood that other methods for comparing the object to a fitting model may be used without departing from the scope of invention.

If the computing device 108 detects the presence of an object (e.g., animal) in the scan, the computing device moves on to validate (304) the posture of the animal appearing in the scan. Determination of an accurate and consistent BCS for an animal necessitates that the animal is in the proper posture when the imaging/scanning device captures an image. If the animal is not in a valid posture, then portions of the animal could be missing from the scan which would lead to an inaccurate or incomplete BCS for the animal.

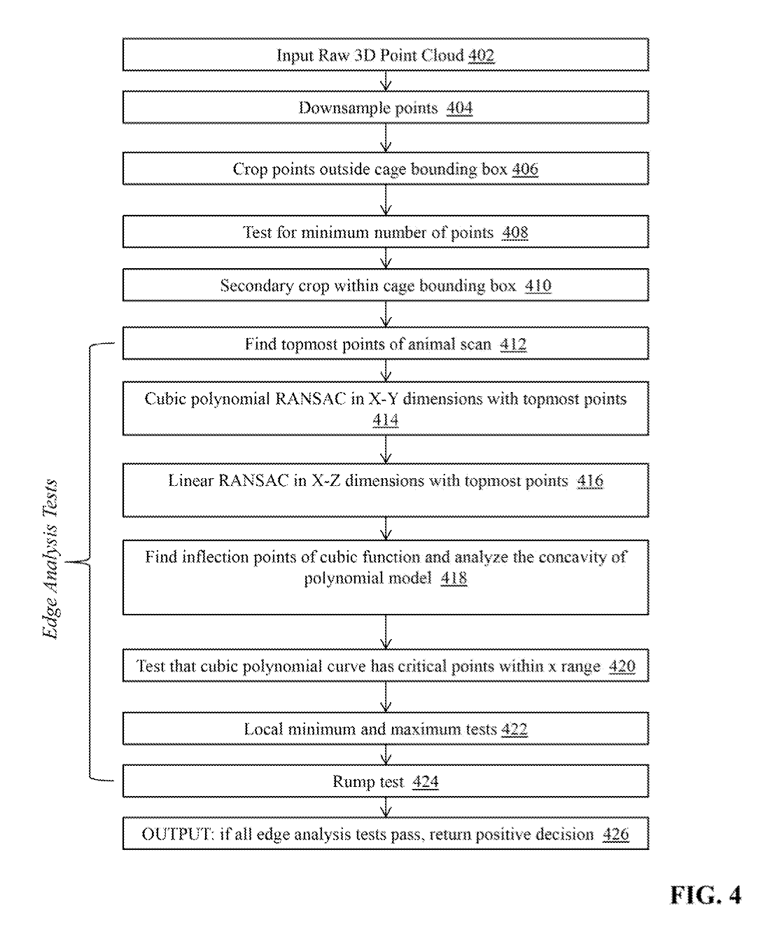

FIG. 4 is a flow diagram of a method for validating the posture of the animal appearing the scan. The computing device 108 receives as input (402) the raw 3D point cloud captured by the imaging/scanning device 104. For example, the raw point cloud can be in a .VDL file format. The computing device 108 also receives the position of the 3D cage, such as the ground and frame sides. In some embodiments, the 3D cage is not used but can help with processing efficiency and consistency. The computing device 108 then downsamples (404) the points in the raw 3D point cloud and crops (406) points outside the boundaries of the 3D cage bounding box to reduce the amount of background noise within the cage boundaries.

The computing device 108 tests (408) the number of points within the cage bounding box to determine whether the number of points exceeds a predetermined minimum value (e.g., to ensure that the scan density is sufficient), and performs a secondary crop (410) of points within the cage bounding box.

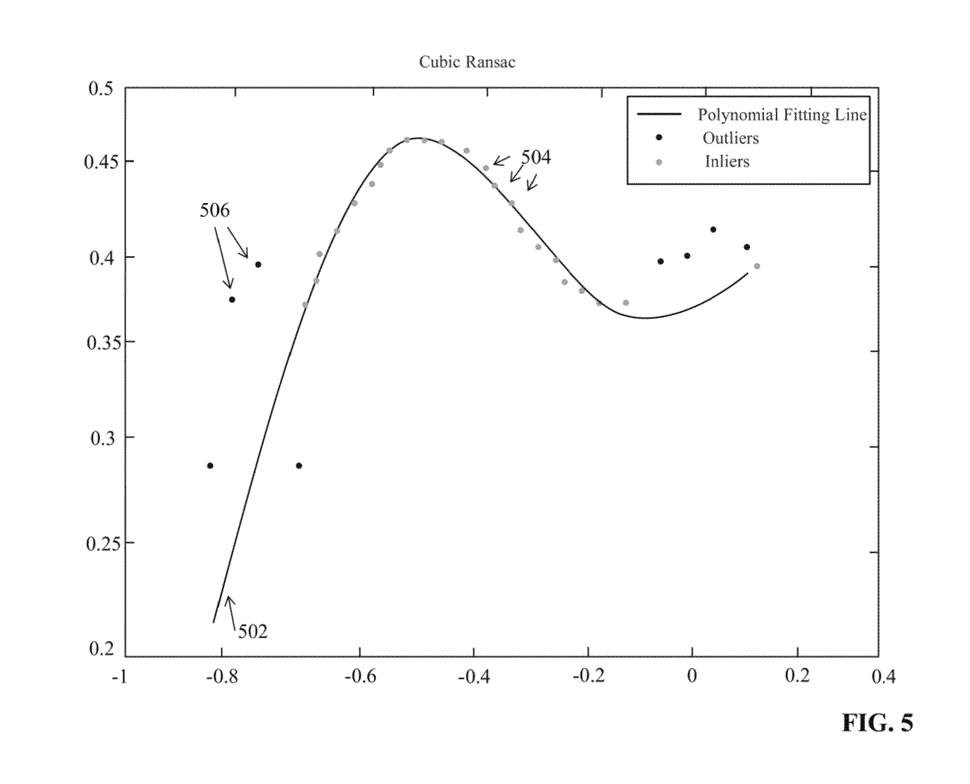

The computing device 108 finds (412) the topmost points of the scan and utilizes a RANSAC algorithm to perform a number of edge analysis tests on the scan (in both a side view and a top-down view) to determine whether the animal's posture is valid. The computing device 108 uses RANSAC to pick a random sample of four points to fit a cubic polynomial curve to (step 414). Then, the distance from the curve to each point is calculated to find which points are inliers to the curve model. The cubic polynomial curve procedure is repeated a set number of times with the goal of reducing error, but in some instances the RANSAC algorithm may not reach a decision. In such cases, the algorithm generates an edge analysis error and produces a negative decision with respect to the animal's posture. FIG. 5 is a diagram of an exemplary cubic polynomial curve model generated by RANSAC. As shown in FIG. 5, many of the points (e.g., points 504) are close to the polynomial curve 502 and are deemed inliers, while other points (e.g., points 506) are further away from the curve 502 and are deemed outliers.

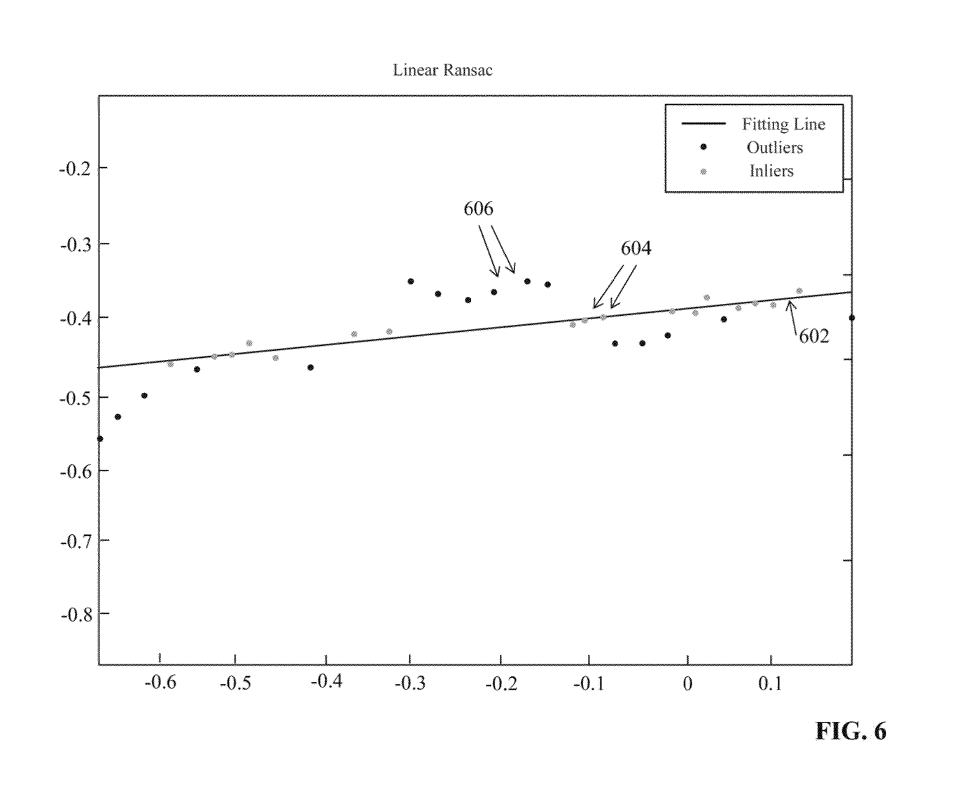

Returning to FIG. 4, the computing device 108 then uses RANSAC to match a “flat” linear model to the X-Z coordinates of the topmost points of the animal's back from a top-down perspective (step 416). A negative decision is automatically produced if the algorithm cannot produce a consensus for the procedure. FIG. 6 is a diagram of an exemplary linear model generated by RANSAC. As shown in FIG. 6, some of the points (e.g., points 604) are close to the fitting line 602, and are deemed inliers. Some of the points (e.g., points 606) are further away from the fitting line 602, and are deemed outliers.

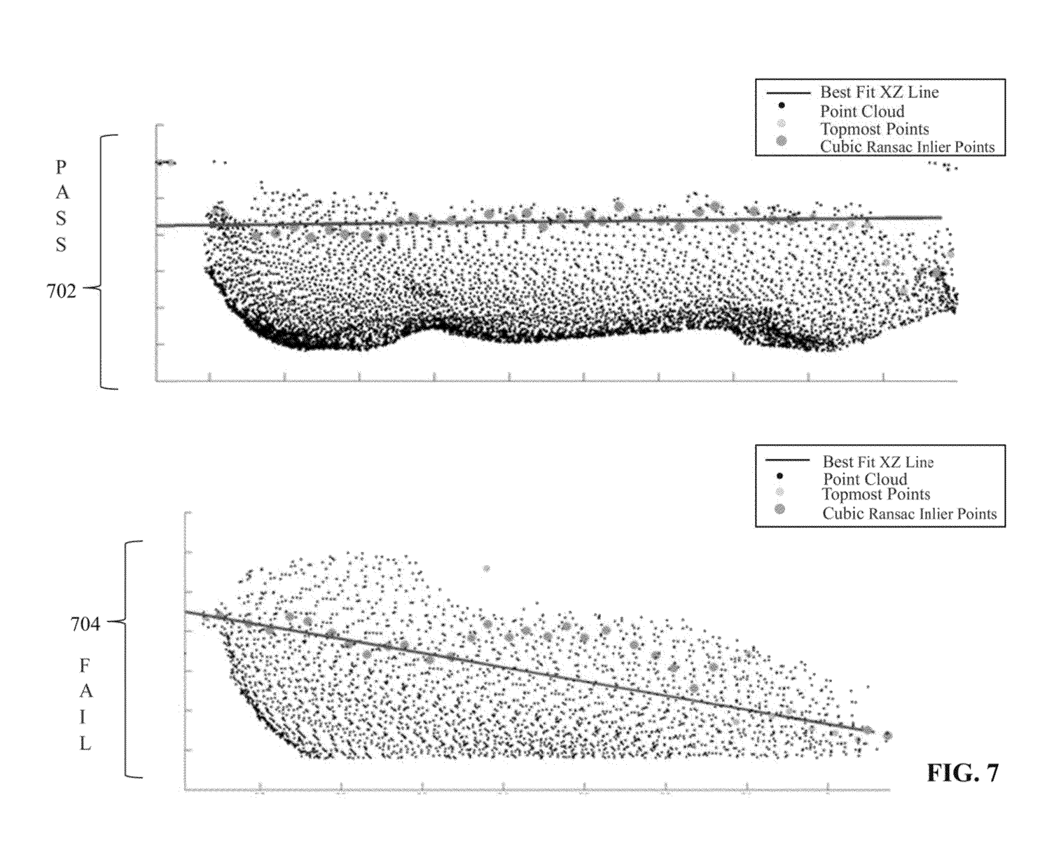

Also, the animal is classified by the algorithm as “bent” if the absolute value of the slope is greater than 0.12, resulting in a negative decision regarding the posture of the animal. FIG. 7 is a diagram of two different results produced by the computing device 108 after conducting the linear model matching. As shown in 702, the fitting line is nearly horizontal and therefore the animal's posture passes the linear model test. However, as shown in 704, the fitting line slopes downward from left to right, producing a failure of the linear model test.

Returning to FIG. 4, the computing device 108 finds (418) inflection points of the cubic polynomial curve and analyzes the concavity of the polynomial curve. These data points are important to determining the posture of an animal as, for example, a hog's back in an upright, standing position produces a back shape that first curves upward then downward.

As modeled by the cubic polynomial curve, the curvature of the topmost points must change within the range of the x values of the point cloud. The computing device 108 tests the value of the inflection point on the polynomial curve, which is where concavity of the curve changes. If the computing device 108 determines that the inflection point is outside of the x range for the point cloud, the computing device 108 returns an edge analysis error and a negative decision to capture a scan of the animal.

Next, the computing device 108 determines whether the concavity of the polynomial curve changes from being concave down on the left-hand side to being concave up on the right-hand side, which the change in concavity occurring at the inflection point. If this concavity change does not occur, then the animal's posture is not valid.

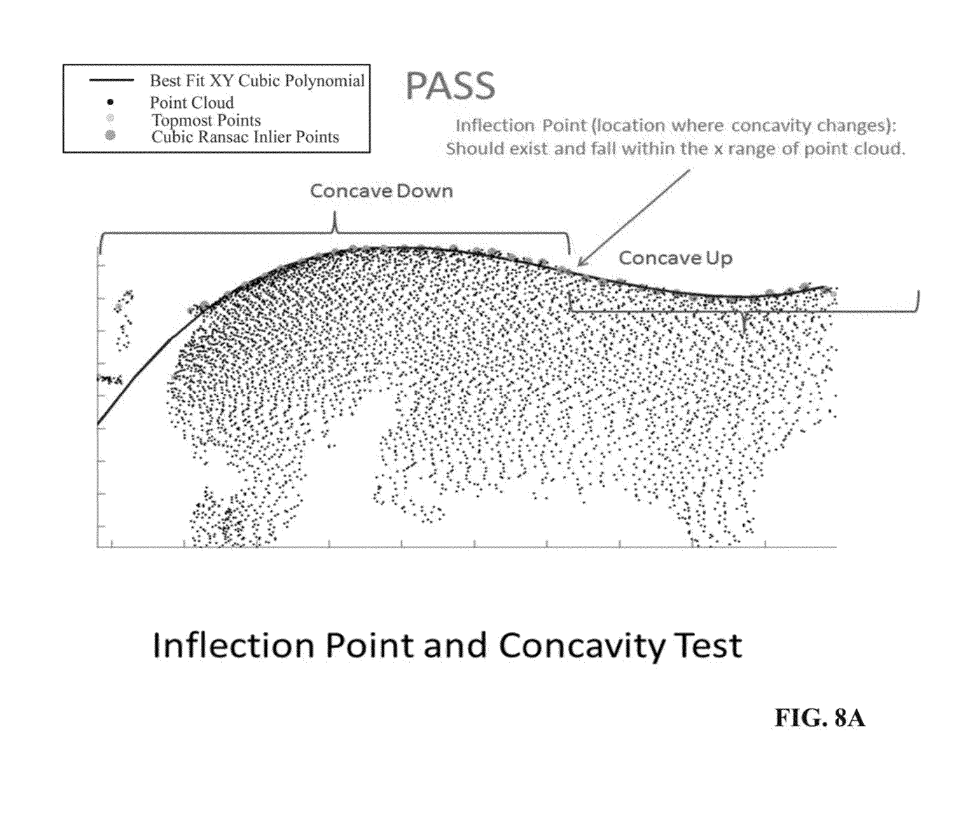

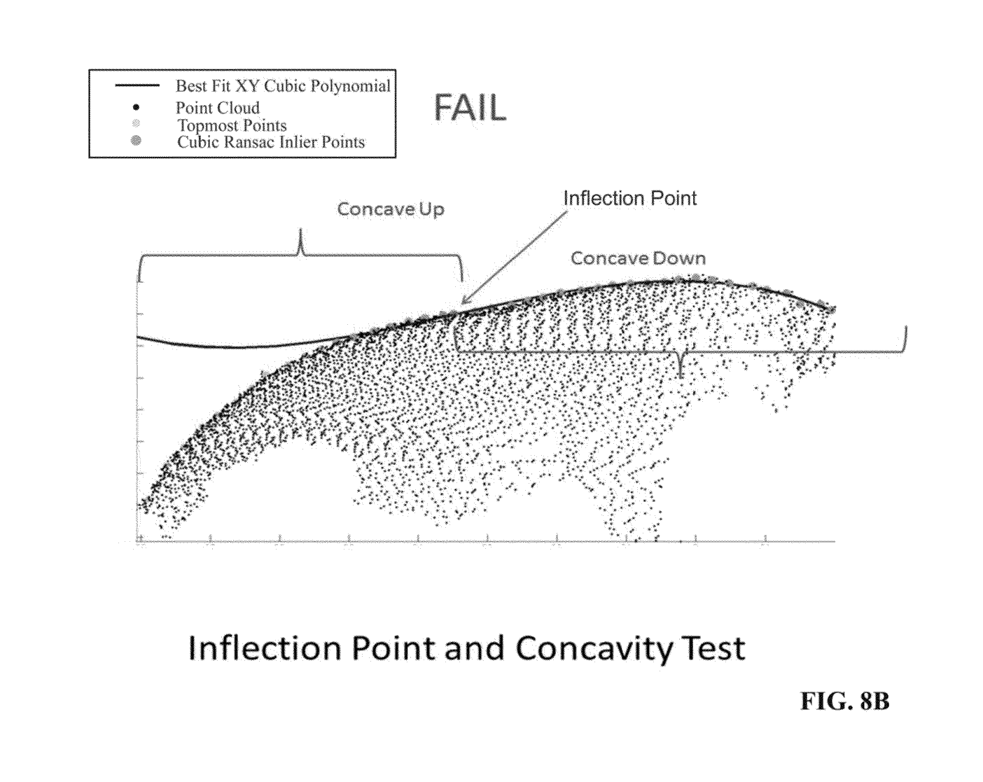

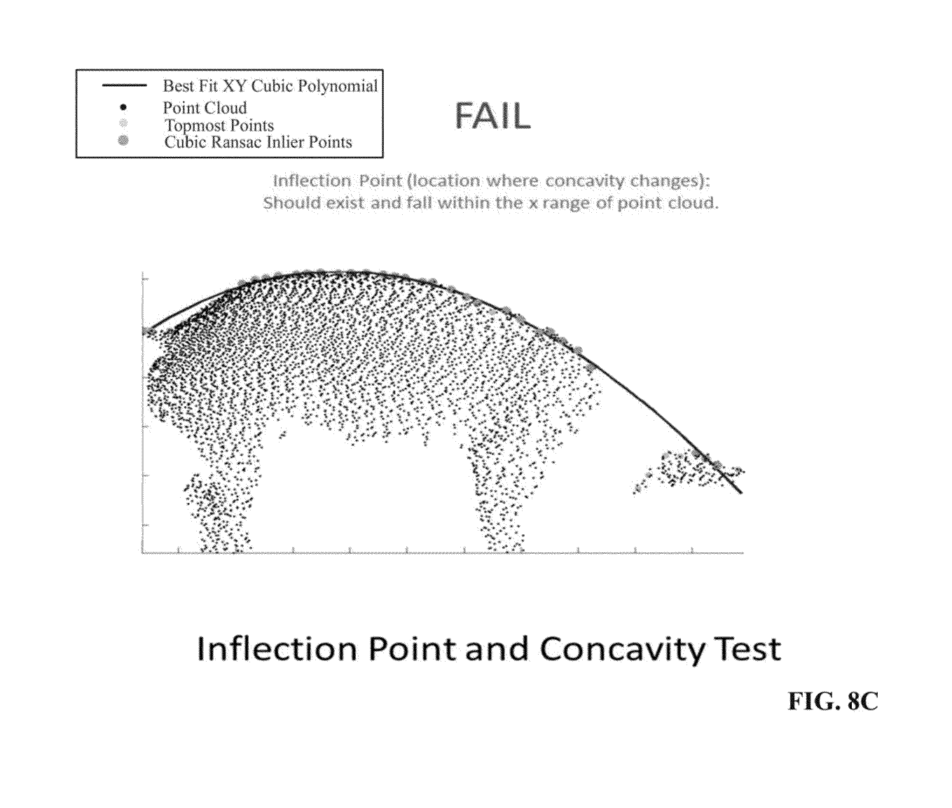

FIG. 8A is a diagram of an exemplary inflection point and concavity test on a cubic polynomial curve where the computing device 108 determined that the inflection point and concavity measurements of the curve pass the test. As shown in FIG. 8A, the inflection point falls within the x range of the point cloud and the left-hand side of the curve is concave down, while the right-hand side of the curve is concave up.

FIG. 8B is a diagram of an exemplary inflection point and concavity test on a cubic polynomial curve where the computing device 108 determined that the concavity measurements of the curve fail the test. As shown in FIG. 8B, although the inflection point falls within the x range of the point cloud, the curve is concave up on the left-hand side before the inflection point and is concave down on the right-hand side after the inflection point.

FIG. 8C is a diagram of an exemplary inflection point and concavity test on a cubic polynomial curve where the computing device 108 determined that the inflection point measurements of the curve fail the test. As shown in FIG. 8C, an inflection point is missing from the curve.

Returning to FIG. 4, the computing device 108 then tests (420) the cubic polynomial curve to determine whether the curve has critical points within the x range of the point cloud. For example, the computing device 108 determines whether the curve includes at least one real critical point. Further, the curve should have only local maxima and minima at critical points. If the curve does not have such local maxima and minima, the cubic function is monotonic and the curve is not the desired shape (e.g., the animal is probably sitting down).

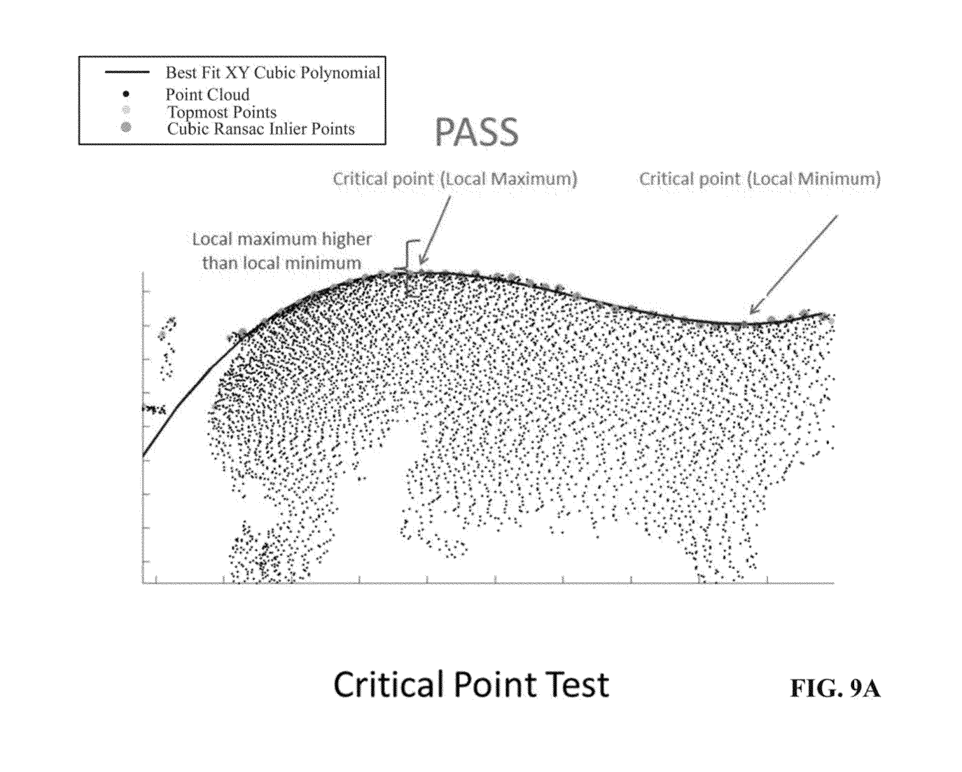

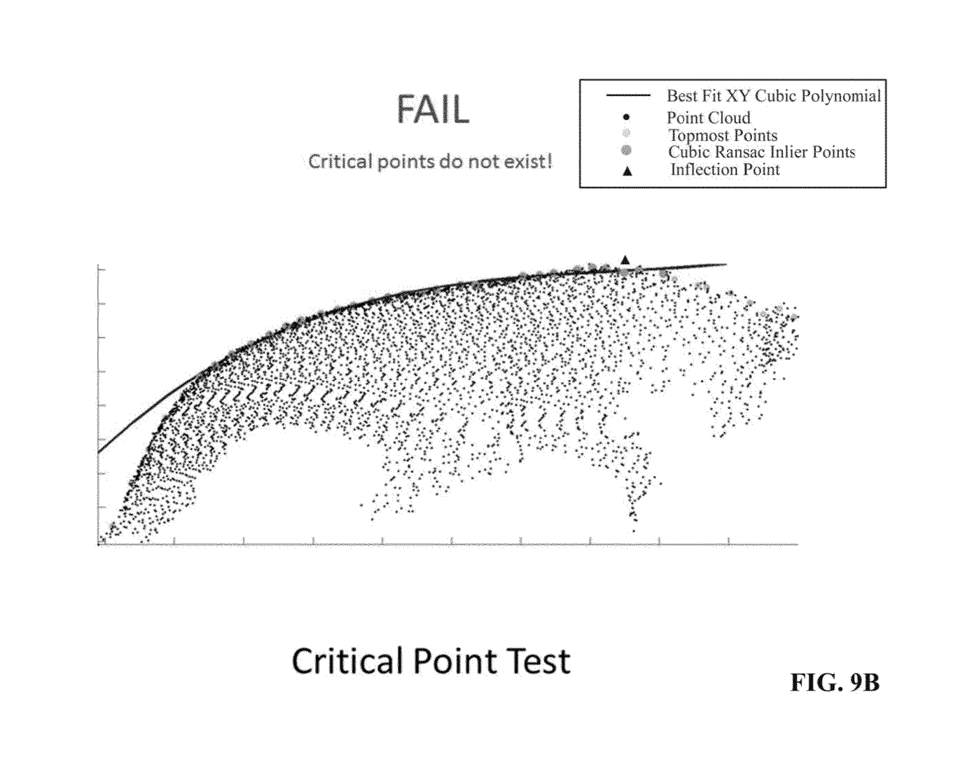

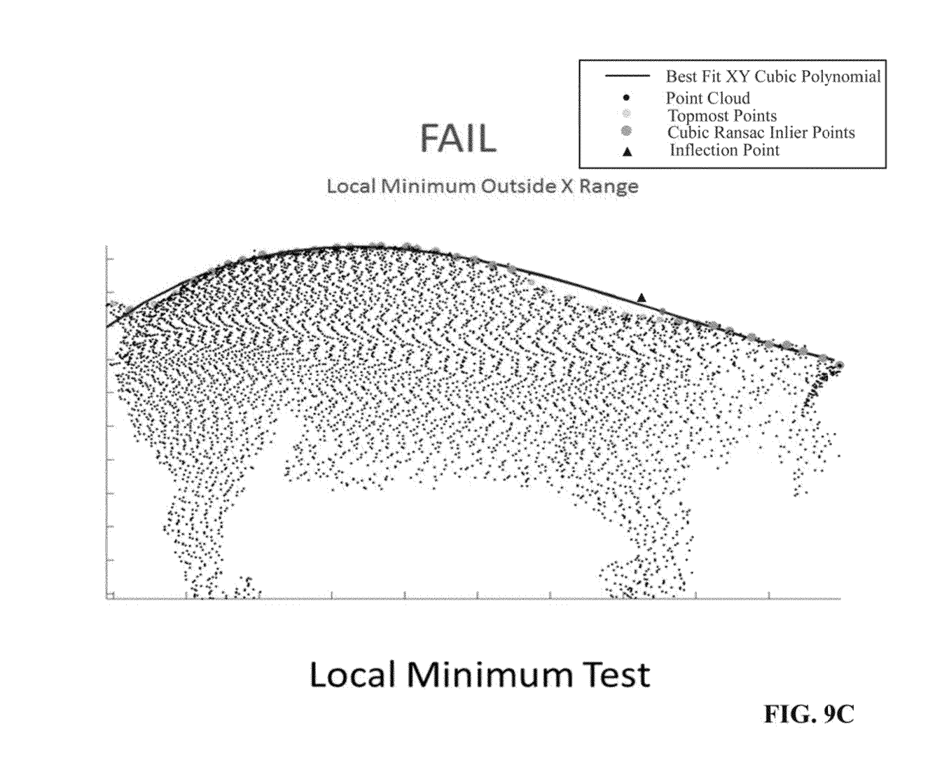

The computing device 108 then tests (422) the curve to determine whether the curve contains a local minimum/minima and a local maximum/maxima, and also whether the local maximum is higher than the local minimum. FIG. 9A is a diagram of an exemplary critical point test on a cubic polynomial curve where the computing device 108 determined that the curve has at least one critical point and that the local maximum and local minimum exist within the x range of the point cloud and the local maximum is higher than the local minimum, thereby passing the test. FIG. 9B is a diagram of an exemplary critical point test on a cubic polynomial curve where the computing device 108 determined that the curve does not have any critical points, thereby failing the test. FIG. 9C is a diagram of an exemplary local minimum test on a cubic polynomial curve where the computing device 108 determined that the local minimum of the curve falls outside the x range of the point cloud, thereby failing the test.

Returning to FIG. 4, the computing device 108 tests (424) the cubic polynomial curve to determine whether the rump point of the curve (the lowest value of the concave downward portion of the topmost points) is higher or lower than the neck point of the curve (the topmost value of the concave downward portion of the topmost points). If the rump point is lower than the neck point, then the test fails.

If the computing device 108 determines positive or passing values for each of the tests in steps 412 through 424, then the computing device 108 concludes that the posture of the animal in the scan is valid and instructs the imaging/scanning device 104 to capture the scan. If the computing device 108 determines a negative or failing value for any of the tests in steps 412 through 424, then the computing device 108 concludes that the posture of the animal is not valid and does not capture the scan.

Once the posture validation procedure is complete and the posture of the animal in the scan has been verified, the computing device 108 crops (306) body parts of the animal as depicted in the scan. The computing device 108 identifies different body parts of the animal that are typically used in BCS calculations, then locates those body parts (also called regions of interest) within the scan and separates the parts for further analysis. For example, the computing device 108 can individually crop regions using any number of 3D cropping algorithms that take a particular 3D area and crop out the points outside the box or defined region.

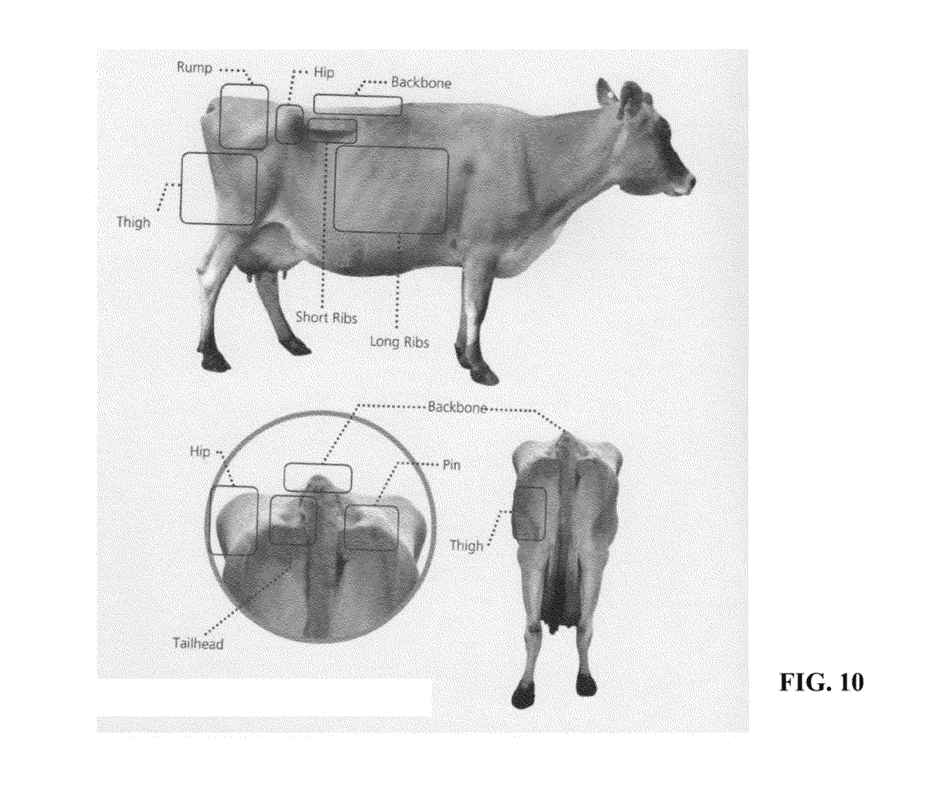

In some embodiments, the body region(s) to be cropped are determined by the type of animal scanned, the breed of the animal, and the sex of the animal. For example, for a cow, a body region can be a rump, hip, backbone, thigh, short-rib, long-rib, tail-head or pin bone. For a pig or hog, the body region can be a backbone, rib, or hip. Further, there are significant differences between the shapes of different breeds and sex of the animals, e.g., a cow, heifer, bull, or steer. A model that may work for a cow does not work for a bull, and a model that works for a male animal may not work for a female animal. Multiple body regions can be cropped from the scan. FIG. 10 is a diagram of exemplary body regions of an animal (e.g., a cow) to be identified by the computing device for determination of a BCS. In some embodiments, a body region can be larger areas of the animal like a side scan or a back scan.

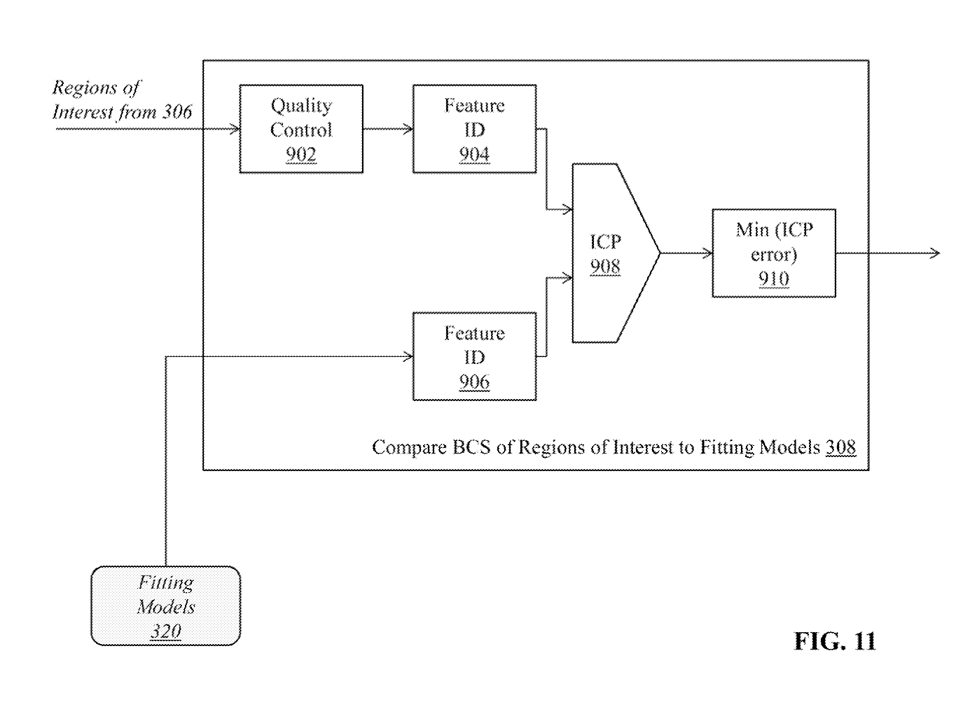

Once the cropped regions of interest have been generated by the computing device 108, the device 108 compares (308) the regions of interest to predefined fitting models (with already known BCS values). FIG. 11 is a flow diagram of a method for comparing the scan of a region of interest for an animal with a fitting model for that region of interest to determine a BCS. The computing device 108 first performs a quality control procedure (step 902) on each of the cropped body region scans to confirm that the scan has adequate point density (e.g., no holes) as well as to determine whether the scan shows any bending or stretching larger than a predefined tolerance level. The computing device 108 then performs a feature identification procedure (step 904) on the cropped body region scans to find certain features within the body region (e.g., a shoulder bone, a tail bone). The features serve as points of reference when comparing the cropped scans to the fitting models.

The computing device 108 retrieves a number of fitting models from database 110 for use in the comparison step. The database 110 can be structured to include many different fitting models for a single type of animal. For example, for a Jersey Cow, the database 110 may include fitting models corresponding to a variety of different body regions (e.g., rump, hip, backbone), and for each body region, the database 110 may include a fitting model for a range of different BCS values (e.g., BCS of one, BCS of two, BCS of ten). The fitting models within the database 110 can be generated by manually evaluating live animals to determine a BCS and then capturing scans of those animals and categorizing the scans.

The structure provided below is an example of how the database 110 can be structured for a Jersey Cow:

| JERSEY | BCS | BCS | BCS | BCS | BCS | BCS | BCS | BCS | BCS | BCS |
|---|---|---|---|---|---|---|---|---|---|---|
| COW | One | Two | Three | Four | Five | Six | Seven | Eight | Nine | Ten |
| Rump | FM | FM | FM | FM | FM | FM | FM | FM | FM | FM |
| Hip | FM | FM | FM | FM | FM | FM | FM | FM | FM | FM |
| Backbone | FM | FM | FM | FM | FM | FM | FM | FM | FM | FM |
| Ribs | FM | FM | FM | FM | FM | FM | FM | FM | FM | FM |
| Thigh | FM | FM | FM | FM | FM | FM | FM | FM | FM | FM |
| Pin | FM | FM | FM | FM | FM | FM | FM | FM | FM | FM |
| Tailhead | FM | FM | FM | FM | FM | FM | FM | FM | FM | FM |

As shown in the table above, there are seven different body regions defined for the Jersey Cow and, for each body region, the table stores a fitting model (FM) for an animal that has a known BCS value of one through ten—resulting in a total of seventy different fitting models available.

The computing device 108 retrieves a fitting model corresponding to the same region as the cropped body region scan, and compares the body region scan to the fitting model using an iterative closest point (ICP) methodology 908. The computing device 108 determines an ICP error for points around the corresponding features (e.g., bones) that were previously identified in both the fitting model and the cropped scan, thereby increasing the efficiency of the ICP algorithm by focusing on the relevant points.

In some embodiments, the selected fitting model(s) are adjusted for at least one of a height, a length or a depth of at least one cloud point of (i) the image relative to the fitting model or (ii) the fitting model relative to the image. A differential adjustment parameter for the adjustment of the at least one cloud point is calculated, a size of the animal is determined by adjusting the known size of the model based upon the differential adjustment parameter.

For example, a model can be adjusted in both height-width-depth directions until it is closest to the cropped animal in the scan. The model is adjusted by a ratio (e.g., R1) so the model is as close to the size of the scan as possible. The idea here is to “fit” the model to the scan the best it can in the X-Y-Z direction. The size can be further refined by using additional steps to check for adjustments in each of the X-, Y-, and Z-axis which correspond to height, width, and thickness of the animal using a similar approach.

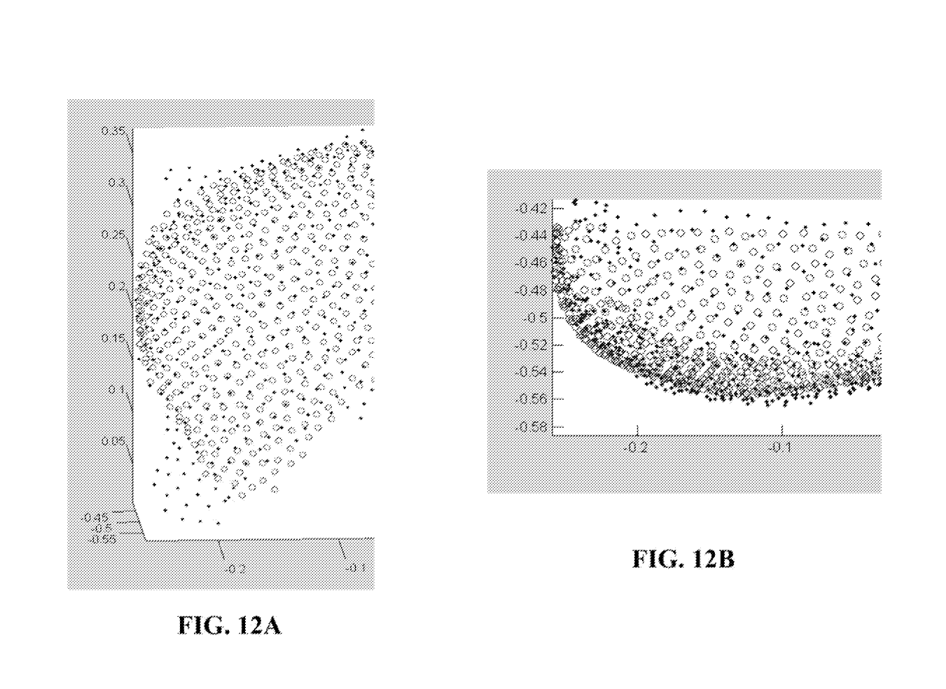

FIG. 12A is a side view diagram of a comparison between a 3D scan and a fitting model using an iterative closest point algorithm. As shown in FIG. 12A, the solid dots represent the actual 3D scan taken by the imaging/scanning device 104 and sent to the computing device 108, while the small circles represent the fitting model. When the computing device 108 runs the iterative closest point algorithm on the 3D scan and the fitting model, the scan and the model are compared to determine a characteristic (e.g., fatness) of the animal relative to the fitting model. FIG. 12B is a top-down view diagram of the comparison in FIG. 12A between a 3D scan and a fitting model using an iterative closest point algorithm.

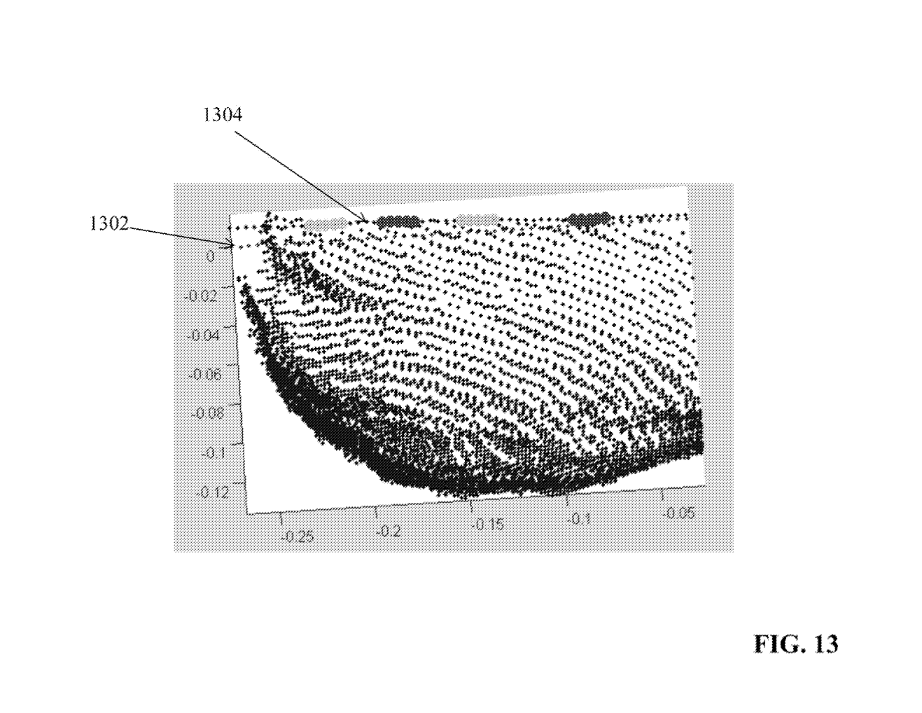

FIG. 13 is a diagram of a comparison between the half-lines of a 3D scan of an animal and a fitting model. The half-line of the fitting model 1302 is compared to the half-line of the 3D scan 1304. As seen in FIG. 13, the captured 3D scan is “fatter” than the fitting model. Hence, the computing device 108 continues to look for a fitting model of larger BCS (or “fatter”) in order to determine the best fit. As described above, the computing device 108 continues this process until the computing device 108 determines a minimum ICP error occurs between the 3D scan and the fitting model.

Once the computing device 108 has determined that a particular fitting model has the minimum ICP error 910, that fitting model is considered the “best fit” and the BCS value for the fitting model is assigned to the cropped body region scan. For example, if the computing device 108 determines that the fitting model for the rump region having a known BCS value of six produces the minimum ICP error with respect to a cropped scan of a rump, then the cropped scan is assigned a BCS value of six. When two fitting models return the same minimum ICP error, the computing device 108 can average the BCS value for those fitting models and assign the average value to the cropped scan.

After the computing device 108 has performed the ICP algorithm 908 to compare each of the scanned body regions with the fitting models and determined a BCS value for each body region scan, the computing device 108 then determines (310) the overall BCS value for the animal. There are many techniques within the scope of the invention that can be used to determine the overall BCS value. In one example, the computing device 108 simply averages the BCS values for each of the body regions and determines an overall BCS. In another example, the computing device 108 assigns a weight to each body region and then averages the BCS values based on the weight. For example, a shoulder could have a weight of one, whereas a rump could have a weight of two. If the BCS for the shoulder is five and the BCS for the rump is seven, then the overall BCS value is (1×5+2×7)/3=6.3. Other types of algorithms, such as machine learning with training data, can be used.

The above-described systems and methods can be implemented in digital electronic circuitry, in computer hardware, firmware, and/or software. The implementation can be as a computer program product (e.g., a computer program tangibly embodied in an information carrier). The implementation can, for example, be in a machine-readable storage device for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the technology by operating on input data and generating output. Method steps can also be performed by and an apparatus can be implemented as special purpose logic circuitry. The circuitry can, for example, be a FPGA (field programmable gate array) and/or an ASIC (application-specific integrated circuit). Modules, subroutines, and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data.

Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device, a transmitting device, and/or a computing device. The display device can be, for example, a cathode ray tube (CRT) and/or a liquid crystal display (LCD) monitor. The interaction with a user can be, for example, a display of information to the user and a keyboard and a pointing device (e.g., a mouse or a trackball) by which the user can provide input to the computer (e.g., interact with a user interface element). Other kinds of devices can be used to provide for interaction with a user. Other devices can be, for example, feedback provided to the user in any form of sensory feedback (e.g., visual feedback, auditory feedback, or tactile feedback). Input from the user can be, for example, received in any form, including acoustic, speech, and/or tactile input.

The computing device can include, for example, a computer, a computer with a browser device, a telephone, an IP phone, a mobile device (e.g., cellular phone, personal digital assistant (PDA) device, laptop computer, electronic mail device), and/or other communication devices.

The computing device can be, for example, one or more computer servers. The computer servers can be, for example, part of a server farm. The browser device includes, for example, a computer (e.g., desktop computer, laptop computer, tablet) with a world wide web browser (e.g., Microsoft® Internet Explorer® available from Microsoft Corporation, Mozilla® Firefox available from Mozilla Corporation, Safari available from Apple). The mobile computing device includes, for example, a personal digital assistant (PDA).

Website and/or web pages can be provided, for example, through a network (e.g., Internet) using a web server. The web server can be, for example, a computer with a server module (e.g., Microsoft® Internet Information Services available from Microsoft Corporation, Apache Web Server available from Apache Software Foundation, Apache Tomcat Web Server available from Apache Software Foundation).

The storage module can be, for example, a random access memory (RAM) module, a read only memory (ROM) module, a computer hard drive, a memory card (e.g., universal serial bus (USB) flash drive, a secure digital (SD) flash card), a floppy disk, and/or any other data storage device. Information stored on a storage module can be maintained, for example, in a database (e.g., relational database system, flat database system) and/or any other logical information storage mechanism.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

The above described networks can be implemented in a packet-based network, a circuit-based network, and/or a combination of a packet-based network and a circuit-based network. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

Comprise, include, and/or plural forms of each are open ended and include the listed parts and can include additional parts that are not listed. And/or is open ended and includes one or more of the listed parts and combinations of the listed parts.

While the invention has been particularly shown and described with reference to specific illustrative embodiments, it should be understood that various changes in form and detail may be made without departing from the spirit and scope of the invention.

What is claimed is:

1. A method for determining a body condition score (BCS) for an animal, the method comprising:
capturing, by an imaging device, at least one image of an animal, wherein each image includes a body region of the animal, and transmitting the image to a computing device;
identifying, by a computing device, the body region contained in the image;
cropping, by the computing device, a portion of the image associated with the identified body region from the image;
comparing, by the computing device, the cropped portion of the image with one or more fitting models corresponding to the body region; and
determining, by the computing device, a body condition score for the body region based upon the comparing step.

2. The method of claim 1, further comprising validating, by the computing device, a posture of the animal in the image.

3. The method of claim 2, the validating step further comprising:
generating a 3D point cloud based upon the image;
performing one or more edge analysis tests on the 3D point cloud; and
determining whether the posture is valid based upon the one or more edge analysis tests.

4. The method of claim 3, wherein performing one or more edge analysis tests includes:
generating a cubic polynomial curve based upon the topmost points of the 3D point cloud; and
analyzing the inflection point and concavity of the cubic polynomial curve.

5. The method of claim 4, further comprising analyzing the local minimum and maximum of the cubic polynomial curve.

6. The method of claim 3, wherein performing one or more edge analysis tests includes:
generating a linear model based upon the topmost points of the 3D point cloud; and
analyzing the slope of the linear model.

7. The method of claim 6, wherein the computing device determines that the posture is not valid if the slope of the linear model exceeds 0.12.

8. The method of claim 1, wherein each image includes a plurality of body regions.

9. The method of claim 1, wherein identifying the body region contained in the image includes:
determining a type of the animal in the image; and
selecting a portion of the image corresponding to the body region based upon the animal type.

10. The method of claim 9, wherein the animal type includes a sex of the animal, a breed of the animal, and a species name of the animal.

11. The method of claim 1, wherein comparing the cropped portion of the image with one or more fitting models corresponding to the body region includes:
retrieving the one or more fitting models from a database based upon a type of the animal and the body region;
identifying one or more anatomical features in the cropped portion of the image;
identifying one or more anatomical features in the fitting models; and
comparing cloud points in the cropped portion of the image that are associated with the one or more anatomical features with cloud points in the one or more fitting models that are associated with the one or more anatomical features.

12. The method of claim 11, wherein each of the one or more fitting models is associated with a known BCS.

13. The method of claim 11, wherein the comparing cloud points step includes:
determining an error value between the cloud points in the cropped portion of the image and the cloud points in the one or fitting models; and
selecting the fitting model that has the minimum error value.

14. The method of claim 13, wherein the determining an error value step is based upon an iterative closest point algorithm.

15. The method of claim 13, further comprising assigning the BCS associated with the fitting model that has the minimum error value to the cropped portion of the image.

16. The method of claim 11, further comprising adjusting at least one of height, length or depth of at least one cloud point of (i) the image relative to one of the fitting models or (ii) one of the fitting models relative to the image.

17. The method of claim 11, further comprising determining whether the density of cloud points in the image meets a predefined threshold.

18. The method of claim 1, wherein the image is a 3D scan.

19. The method of claim 1, wherein the body region is a rump, hip, backbone, thigh, short-rib, long-rib, tail-head, or pin bone of the animal.

20. The method of claim 1, wherein the BCS is a score on a scale that indicates fatness or thinness of the animal.

21. The method of claim 1, wherein each image includes a plurality of body regions, the method further comprising determining an overall body condition score for the animal based upon the body condition score for each of the plurality of body regions.

22. A system for determining a body condition score (BCS) for an animal, the system comprising:
an imaging device configured to capture at least one image of an animal, wherein each image includes a body region of the animal, and transmit the image to a computing device coupled to the imaging device;
the computing device configured to:
identify the body region contained in the image;
crop a portion of the image associated with the identified body region from the image;
compare the cropped portion of the image with one or more fitting models corresponding to the body region; and
determine a body condition score for the body region based upon the comparing step.

23. The system of claim 22, the computing device further configured to validate a posture of the animal in the image.

24. The system of claim 23, the validating step further comprising:
generating a 3D point cloud based upon the image;
performing one or more edge analysis tests on the 3D point cloud; and
determining whether the posture is valid based upon the one or more edge analysis tests.

25. The system of claim 24, wherein performing one or more edge analysis tests includes:
generating a cubic polynomial curve based upon the topmost points of the 3D point cloud; and
analyzing the inflection point and concavity of the cubic polynomial curve.

26. The system of claim 25, further comprising analyzing the local minimum and maximum of the cubic polynomial curve.

27. The system of claim 24, wherein performing one or more edge analysis tests includes:
generating a linear model based upon the topmost points of the 3D point cloud; and
analyzing the slope of the linear model.

28. The system of claim 27, wherein the computing device determines that the posture is not valid if the slope of the linear model exceeds 0.12.

29. The system of claim 22, wherein each image includes a plurality of body regions.

30. The system of claim 22, wherein identifying the body region contained in the image includes:
determining a type of the animal in the image; and
selecting a portion of the image corresponding to the body region based upon the animal type.

31. The system of claim 30, wherein the animal type includes a sex of the animal, a breed of the animal, and a species name of the animal.

32. The system of claim 22, wherein comparing the cropped portion of the image with one or more fitting models corresponding to the body region includes:
retrieving the one or more fitting models from a database based upon a type of the animal and the body region;
identifying one or more anatomical features in the cropped portion of the image;
identifying one or more anatomical features in the fitting models; and
comparing cloud points in the cropped portion of the image that are associated with the one or more anatomical features with cloud points in the one or more fitting models that are associated with the one or more anatomical features.

33. The system of claim 32, wherein each of the one or more fitting models is associated with a known BCS.

34. The system of claim 32, wherein the comparing cloud points step includes:
determining an error value between the cloud points in the cropped portion of the image and the cloud points in the one or fitting models; and
selecting the fitting model that has the minimum error value.

35. The system of claim 34, wherein the determining an error value step is based upon an iterative closest point algorithm.

36. The system of claim 34, the computing device further configured to assign the BCS associated with the fitting model that has the minimum error value to the cropped portion of the image.

37. The system of claim 32, the computing device further configured to adjust at least one of height, length or depth of at least one cloud point of (i) the image relative to one of the fitting models or (ii) one of the fitting models relative to the image.

38. The system of claim 32, the computing device further configure to determine whether the density of cloud points in the image meets a predefined threshold.

39. The system of claim 22, wherein the image is a 3D scan.

40. The system of claim 22, wherein the body region is a rump, hip, backbone, thigh, short-rib, long-rib, tail-head, or pin bone of the animal.

41. The system of claim 22, wherein the BCS is a score on a scale that indicates fatness or thinness of the animal.

42. The system of claim 22, wherein each image includes a plurality of body regions, the computing device further configured to determine an overall body condition score for the animal based upon the body condition score for each of the plurality of body regions.
